(19) United States
(12) Patent Application Publication
(10) Pub. No.: US 2026/0207822 A1
(43) Pub. Date: Jul. 23, 2026

(54) USE OF BONE GRAFT PARTICLES FOR THE PREPARATION OF A POROUS BONE GRAFT MATERIAL

(71) Applicants: INSTITUT STRAUMANN AG, Basel (CH); BIOMIMETIC INNOVATIONS LIMITED, Clare (IE)

(72) Inventors: Benjamin BELLON PECECNIK, Basel (CH); Ole ZOFFMANN ANDERSEN, Fredericia (DK); Benjamin PIPPENGER, Hegenheim (FR)

(21) Appl. No.: 19/140,193

(22) PCT Filed: Dec. 20, 2023
(86) PCT No.: PCT/EP2023/087139

§ 371 Date: Jun. 17, 2025

(30) Foreign Application Priority Data
Dec. 21, 2022 (EP) 22215368.6

(51) Int. Cl. A61L 27/46; A61L 27/22; A61L 27/56

(52) U.S. Cl. CPC A61L 27/46; A61L 27/227; A61L 27/56; A61L 2300/112; A61L 2430/02

(57) ABSTRACT

The use of bone graft particles for the preparation of a porous bone graft material including an acid containing self-setting adhesive composition. The bone graft particles have a porous core with an outer surface which is at least partially covered by an outer layer. The core includes a core material selected from the group consisting of an alloplast, a bone xenograft, and a bone allograft, or a mixture thereof. The outer layer of the particles includes a carbonate salt.

The present invention relates to the use of bone graft particles for the preparation of a porous bone graft material.

Conventionally, regeneration is achieved by filling a bone repair site with a bone graft. Over time, the bone graft is incorporated by the host and new bone remodels the bone graft.

Bone grafts may be alloplast, xenograft, autograft or allograft with similar mechanical properties to bone.

In oral surgery and orthopedics, synthetic bone repair materials on a hydroxyapatite (HA) and/or tricalcium phosphate (TCP) basis are widely used. Depending on indications, they may be applied as granules or prefabricated blocks. U.S. Pat. No. 6,511,510 relates to a porous ceramic material from calcium phosphates obtained by a sintering process. The use of granular material allows treatment of a wide range of indications. For granular material, the ceramic block material is processed by steps such as rubbing, pounding and sieving afterwards (WO 04/054633). Although the granular materials are applied to a wide range of indications in terms of size and area, their suitability to treat large bone defects is limited, because they tend to migrate and as a result to be encapsulated. The augmented volume defined by the applied granules may collapse and fail to guide regrowth of the bone to its original dimensions. U.S. Pat. No. 7,012,034 describes a block-shaped bone augmentation material based on porous-tricalcium phosphate.

In a typical periodontal surgical bone repair procedure, an incision is made in the gum tissue to expose a bone defect adjacent to a tooth root. Once the defect and root are debrided, a bone repair material, suspended in a suitable carrier is placed. The gum tissue is then closed, maintaining the repair material in place. Optionally, a barrier material may be utilized to retain the repair formulation in contact with the defect. Therefore, a bone repair material in periodontal surgery requires formulations that can be easily shaped to size and shape of the defect.

WO 2004/011053 suggests a formulation with a putty consistency. Similarly, EP 1 490 123 describes a kneadable and pliable bone replacement material on a granular calcium phosphate and hydrogel basis. When applied to the defect site, the formulation remains adhered thereto without migration or excessive expansion.

US2020147260 discloses an interconnected porous calcium carbonate body in which a sulfate ion component that is an anion component of calcium sulfate serving as a raw material inorganic compound and an anion component of sodium carbonate serving as an electrolyte are exchanged. These particles are combined with other inorganic compounds including phosphoric acid.

US2013122057 discloses cements containing certain small molecule amino acid phosphate compounds such as phosphoserine and certain multivalent metal compounds.

US 2022/023493A1 discloses a sticky composition comprising a multivalent metal salt, an osteoinductive factor and β-TCP granules in an aqueous solution or suspension which hardens in situ. Although said material has excellent adhesion properties, it is very dense and clogs the pores of the β-TCP granules which negatively impacts the osseointegration.

The problem of the present invention was therefore, to provide a bone graft material which allows a good osseointegration.

The problem is solved by the particles according to claim 1. Further preferred embodiments are subject of dependent claims 2 to 15.

Surprisingly, it was found that the bone graft particles according to the present invention are useful for the preparation of a porous bone graft material comprising an acid containing self-setting adhesive composition. They have a porous core with an outer surface which is at least partially covered by an outer layer. Preferably, at least 50% of said outer surface is covered by said outer layer. The core comprises a core material selected from the group consisting of a bone alloplast, a bone xenograft, and a bone allograft, or a mixture thereof and the outer layer comprises a carbonate salt.

As soon as the particles according to the present invention come in contact with the acid containing self-setting adhesive composition, the carbonate salt in the outer layer of the particles starts to form carbon dioxide bubbles which prevent the self-setting adhesive composition from flowing into the pores. In addition, said bubbles also form additional pores in the self-setting adhesive composition during curing, thus forming a porous scaffold. Despite the high porosity, the bone graft material has sufficient stability to prevent movement and it is strong enough to withstand the forces within the implantation site, i.e. is resistant to mechanical stress. Furthermore, due to the sticky character of the acid containing self-setting adhesive composition the particles do not migrate and stay at the site of administration. In addition, due to its putty state the bone graft material is for about 1 to two minutes shapeable and can occupy voids of varying shapes. Thus, the particles according to the present invention allow to prepare a porous bone graft material which ensures stability of grafted sites while also having optimal resorption kinetics and, therefore, osteogenesis.

The use of the coated bone graft particles according to the present invention allows to obtain a bone graft material having a high number of open pores which promotes revascularization, healing, and remodeling of bone. The high number of open pores facilitates the adhesion and invasion of bone forming cells.

The term “carbonate salt” includes salts of primary carbonates, i.e. hydrogen carbonates (HCO₃⁻) and salts of secondary carbonates (HCO₃²⁻).

The term “self-setting” refers to the ability of the material to cure and harden as a result of the mixing of the solid and the liquid component.

The term “bone alloplast” means a synthetic, inorganic, biocompatible bone substitute which contains no animal or human components.

The term “bone xenograft” stands for particles having their origin in the bone tissue of an animal. Preferably, the porous or spongy parts of the joint balls of animals are used to prepare the bone xenograft. Regarding the porosity, the use of animals, preferably grown-up cattle, has been shown to be particularly suitable for extracting the spongy parts of leg and arm joints, since the trabecular structure is fully developed and large portions with the same pore structure are present.

As used herein the term “bone allograft” means bone including cortical and/or cancellous bone, recovered from another individual and processed for implantation into a living patient including for example: fibular wedges; humeral wedges; tibial wedges; fibular trapezoid wedges; humeral trapezoid wedges; femoral trapezoid wedges; fibular shafts and rings; humeral shafts and rings; and femoral shafts and rings; and essentially intact bone grafts including for example proximal and distal femur, femoral head; and small cut bone grafts including for example cancellous cubes, iliac crest wedges, and cloward dowels.

Within the context of the present invention the term “pore” means and includes any void in a material and includes voids of any size and shape. For example, pores include generally spherical voids, generally rectangular voids, as well as elongated voids or channels having any cross-sectional shape including nonlinear or irregular shapes. In particular, the term pore includes nanopores, micropores and macropores as defined below.

The particles according to the present invention stimulate tissue formation and tissue ingrowth and therefore preferably comprise pores at different size ranges from 1 nm to 1500 micrometer, most preferably with an average pore size between 500 nm and 1000 micrometer. Event more preferred 1 micrometer to 500 micrometer. The material thereby may comprise nanopores in the 1 nm to 100 nm range, micropores in the 100 nm to 1 micrometer range as well as macropores larger than 50 micrometer.

Preferably, not only the outer surface of the porous core is at least partially covered by a carbonate salt, but also a least part of the surfaces of the pores. It is believed that any self-setting bioadhesive getting into the pores of the particles will initiate gas formation and, thus, be pushed back out of the pores by the increased pressure.

In one aspect of the present invention, the particles can have a weight ratio of carbonate salt to core material from 15:100 to 1:100, preferably 8:100 to 2:100 and most preferably from 4:100 to 2:100. It could be shown that said ratio has a significant impact on the bubble formation. A ratio of between 4:100 and 2:100 resulted in bigger pores and a substantial volume growth.

Preferably, the core material of the particles according to the present invention is a bone alloplast which is selected from the group consisting of hydroxy apatite, α-tricalcium phosphate, β-tricalcium phosphate, tetracalcium phosphate, dicalcium phosphate anhydrous, dicalcium phosphate dihydrate amorphous calcium phosphate, calcium deficient hydroxyapatite, calcium sulphate and bioactive glass. The advantage of said materials is their biocompatibility, safety, and efficacy in periodontal regeneration and guided bone regeneration.

Preferably, the carbonate salt present in the outer layer of the particles according to the present invention is selected from the group consisting of sodium hydrogen carbonate, ammonium hydrogen carbonate, sodium carbonate, calcium carbonate, strontium carbonate and magnesium carbonate. Said carbonates are all biocompatible. Best results could be obtained with a carbonate selected from the group consisting of sodium hydrogen carbonate, ammonium hydrogen carbonate, sodium carbonate, and in particular with sodium hydrogen carbonate. They have all a good solubility in water. This allows a fast and cheap coating of the particles, for example, by mixing them with an aqueous solution comprising said carbonate salts. Preferably, the carbonate concentration is adjusted depending on the core material to prevent clogged pores in the final product.

In one aspect of the present invention the core of the particles has a porosity of at least 50%, preferably at least 60% and most preferably of 65 to 80%. Due to the presence of the carbonate salt on its surface and optionally also on the surface of the pores, said pores remain open which allows to wick, soak, and imbibe blood very quickly which is a key requirement for a good osseointegration.

In one embodiment of the present invention the porosity occurs in a broad range of effective pore sizes. The particles may have, at once, nanoporosity, microporosity and macroporosity. It is preferred that nano-, micro and macroporosity occur simultaneously and may be interconnected. In a preferred embodiment of the present invention the particles have a mean pore size of 200-1000 μm, preferably of 600-900 μm which results in an excellent osseointegration.

Porosity can be measured by Helium Pycnometry. This procedure is known to the skilled person and determines the density and true volume of a sample by measuring the pressure change of helium in a calibrated volume.

Preferably, the bone graft particles have an average particle size of at 0.1 to 3.0 mm, preferably 0.5 to 2.0 mm. Preferably particles with an average particle size of 0.5 to 1.0 mm are used for smaller bone defects. Use of said particles results in better surface contouring, especially in the esthetic region. Particles with an average particle size of 1.0-2.0 mm are preferably used for larger bone defects, since they enable a better revascularization.

Preferably, the acid containing self-setting adhesive composition comprises an aqueous solution and a self-setting powder which comprises at least two different ingredients, that is, a multivalent metal salt and phosphoserine.

Preferably, the multivalent metal salt contained in the self-setting adhesive powder comprises tetracalcium phosphate or tricalcium phosphate, preferably α-tricalcium phosphate. α-TCP is more soluble in the body's bone material which can increase its absorption rate and shortens the healing process.

Preferably, the phosphoserine is present in an amount from 20% to 50% by weight based of the self-setting adhesive powder, preferably, 20 to 30% by weight of the self-setting adhesive powder.

Preferably, the multivalent metal salt is present in an amount from 50 to 90% by weight based of the self-setting adhesive powder, preferably, 70 to 80% by weight of the self-setting adhesive powder.

A further aspect relates to a method for preparing a porous bone graft material by mixing an acid containing self-setting adhesive composition with the particles according to the present invention at the onset of the curing process, i.e. at the beginning of the curing reaction. As mentioned before, the self-setting adhesive composition comprises an aqueous solution and a self-setting adhesive powder. The particles according to the present invention may be premixed with said self-setting adhesive powder before adding the aqueous solution or they may be added directly after mixing the aqueous solution and the self-setting powder. As soon as the aqueous solution and the self-setting adhesive powder come in contact the curing starts. The self-setting adhesive composition has an initial tacky phase, during which time it has adhesive properties, which lasts 1-3 min after which the material becomes putty like. During this phase, carbon dioxide bubbles are formed on the surface of the particles and optionally on the surface of the pores of the porous core of said particles. Said bubbles prevent the self-setting adhesive composition from entering into the pores of the particles. In addition, said bubbles also form additional pores in the self-setting adhesive composition during curing, thus forming a porous scaffold. During the same time limited manipulation of the bone graft material allows removal of excess. The bone graft material reaches full initial set at around 15 min from mixing. However, the material continues to cure and harden thereafter, being 90% complete within 24 h.

Such a bone graft material can withstand tensile and shear bond stresses as high as 3 MPa, similar to the strength of human cancellous bone. Thanks to the particles according to the present invention the scaffold is highly osteoconductive and bioactive, leading to the eventual replacement of bone graft material with new bone. Furthermore, over time, load bearing responsibility is transferred to the new tissue such that mechanical integrity is maintained.

A further aspect of the present invention relates to a kit for preparing a porous bone graft material comprising
a) a first component A comprising an aqueous solution, and
b) a second component B comprising an acid containing self-setting adhesive powder, said self-setting adhesive powder preferably comprising at least a multivalent metal salt and phosphoserine, and
c) a third component C comprising the particles according to the present invention.

In said kit, component A, component B and component C are stored in separate compartments, thus they are physically separated from each other. The separation of the three compartments allows to provide a ready-to-use system having a long pot life.

Alternatively, the kit may comprise only two components, i.e.
a) a first component A comprising an aqueous solution, and
b) a second component B comprising an acid containing self-setting adhesive powder and the particles according to the present invention, wherein said self-setting adhesive powder preferably comprises at least a multivalent metal salt and phosphoserine.

Thus, in this embodiment the acid containing self-setting adhesive powder and the particles are premixed as component B, which is physically separated from the liquid component A, i.e. the aqueous solution, thus, they are stored in two different compartments.

Both kits allow an in situ preparation of the porous bone graft material directly before use. Thus, the separate components are physically separated from each other and are not mixed until directly before use.

Preferably, the acid containing self-setting adhesive composition comprises phosphoserine, and most preferably l-phosphoserine as acid. L-phosphoserine is a component of many endogenous proteins, in particular osteopontin (bone sialoprotein) and is a normal metabolite found in human biofluids. It has a high affinity for bonding to poorly crystalline apatite, suggesting it plays an important role in mineralization processes.

Within the context of the present invention the term aqueous solution means water that may additionally comprise an additive such as a salt. Preferably, it is distilled water or a solution comprising water and saline (0.9% by weight NaCl in water).

In a further aspect of the present invention component A of the kit additionally comprises an acidifying agent selected from the group consisting of hydrochloric acid, ascorbic acid, citric acid, malic acid, tartaric acid, maleic acid, succinic acid, fumaric acid, acetic acid, formic acid and propionic acid or mixtures thereof, preferably citric acid. The presence of an acidifying agent enhances the in situ formation of carbon dioxide on the surface of the particles and leads therefore to a higher porosity of the final product.

Preferably, the multivalent metal salt contained in the kit according to the present invention comprises tetracalcium phosphate or tricalcium phosphate, preferably α-tricalcium phosphate.

Preferably, component B of the kit additionally comprises calcium silicate which also promotes osteogenesis.

The porous bone graft material according to the present invention is preferably used in the treatment of bone defects, such as a void, a gap or a crack, so as to fill the bone defect. Preferably, the bone defect is a large bone defect, since said bone defects are particularly difficult to treat with conventional bone graft materials. In one embodiment of the present invention the bone defect selected from the group consisting of alveolar ridge augmentation and extraction socket restoration. In another embodiment of the present invention the bone defect is selected from the group consisting of calvarial defects, osteochondral defects, fractured vertebrae, intervertebral spinal fusion posterolateral spinal fusion and illiac crest defect repair.

A further aspect of the present invention relates to a method for treating bone defects by applying the porous bone graft material directly after mixing of the acid containing self-setting adhesive composition and the particles according to the present invention to the site of the bone defect, thereby repairing the bone defect.

According to some embodiments of the invention, the method of repairing a bone defect further includes shaping the porous bone graft material in the site of the bone defect, which can be done for example with a spatula.

According to some embodiments of the invention, the method of repairing a bone defect further includes allowing the porous bone graft material to set and cure, to thereby form a cured bone graft material.

### EXAMPLES

### Preparation of Saturated sodium hydrogen carbonate Solution

4 g sodium hydrogen carbonate was added to 10 ml water in a falcon tube and allowed to dissolve while agitating the container (shaking by hand). The solution was left for 30 min and then agitated again to make sure that as much sodium hydrogen carbonate as possible was dissolved in the water. Then the falcon tube was left until the non-dissolved sodium hydrogen carbonate set on the bottom of the tube. For coating of the particles only the saturated solution, without the non-dissolved sodium hydrogen carbonate was used.

### Coating of the Particles

0.27 g Cerabone particles supplied by Straumann, Basel, were added to a 1.5 ml Eppendorf tube. 250 μl saturated sodium hydrogen carbonate solution was added such as the liquid just covered the particles. The Eppendorf tube was then left open and put at 55° C. over night to evaporate the water. What was left were dry Cerabone particles coated with sodium carbonate.

### Mixing With Self-Setting Adhesive

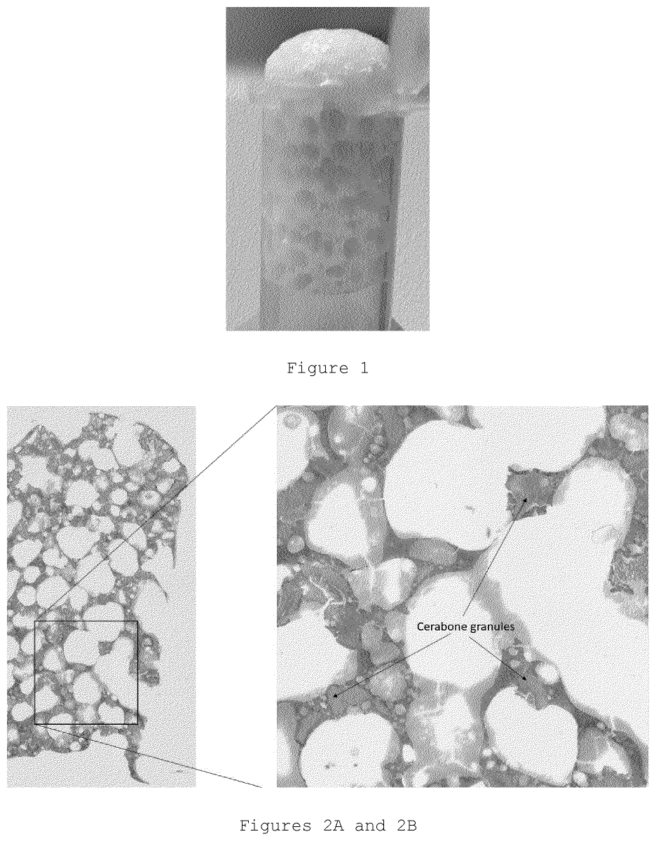

0.5 g of a self-setting adhesive powder comprising 76% α-TCP (Innotere GmbH) and 24% phosphoserine (Merck) was mixed with 200 μl citric acid solution (15%) using a spatula. As soon as the mixture was homogeneous, the particles were added while still mixing with the spatula, to evenly distribute the particles. The mixture was then transferred to an Eppendorf tube (See FIG. 1) and allowed to cure.

### Results

Pore formation is initiated immediately after mixing the self-setting adhesive with the particles. In the section cuts (FIGS. 2A and 2B) it can be seen that the particles are surrounded by pores or immediately adjacent to a pore.

### Experiment 2: Sodium carbonate vs sodium hydrogen carbonate

Instead of a saturated solution the particles were impregnated with a solution comprising three different concentrations

3 different concentrations stock solutions comprising NaHCO₃ were prepared:
96 g/L
48 g/L
24 g/L

3 different concentrations stock solutions comprising Na₂CO₃ were prepared:
96 g/L
48 g/L
24 g/L

For the preparation of the impregnated particles 0.27 g of Cerabone (by botiss) was placed in 1.5 mL Eppendorf tubes and 250 μL of stock solution was added. Subsequently, the prepared samples were left at room temperature, with the lid open, to allow for the water to evaporate.

The following observations were made:

The optimal amount of water for the preparation of the mixtures comprising the particles and the self-setting adhesive powder was increased compared to i comprising self-setting adhesive powder. The optimal amount of water for the NaHCO₃ particles was found to be 175 μl to compensate for the water “consumption” of the particles. The optimal amount of water for the Na₂CO₃ particles was found to be 200 μl to compensate for the water consumption of the particles.

During the experiment it was found that the amount of NaHCO₃ and Na₂CO₃ had a surprising relation to the bubble formation. This was found to be associated with the hydroscopic nature of the particles. The more carbonates that were deposited onto the surface of the particles (i.e. the more concentrated the stock solution was) the more the samples dried out during curing. As a result, particles coated with larger amounts of carbonates appeared drier, during the setting period and this resulted in the formation of smaller pores and less volume growth.

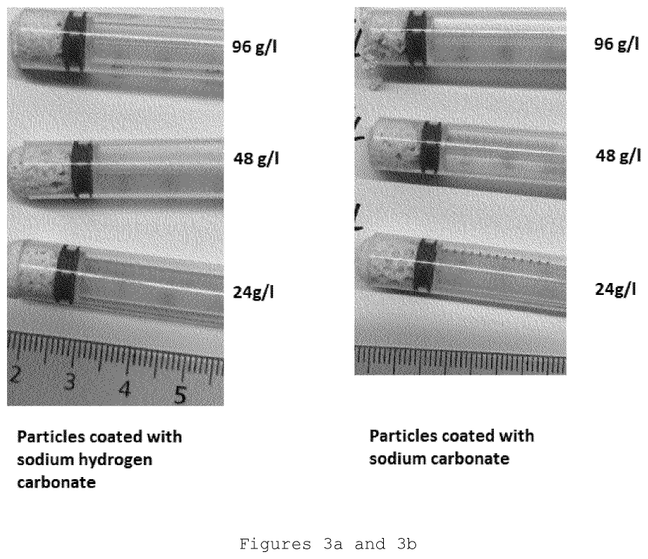

For particles coated with Na₂CO₃ and the self-setting adhesive powder prepared with 200 μl of water, the most optimal porosity was obtained with the low carbonate concentration (24 g/L) and this was also found to be the overall best sample (FIG. 3a). For particles coated with NaHCO₃/and the self-setting adhesive powder with 175 μl of water, the most optimal concentration was found to be the medium concentration (48 g/L). However, the differences were not as distinct as with the Na₂CO₃ samples (FIG. 3b).

1. A porous bone graft material produced by mixing bone graft particles with an acid containing self-setting adhesive composition, wherein:
the bone graft particles include a porous core with an outer surface which is at least partially covered by an outer layer,
the core comprises a core material selected from the group consisting of an alloplast, a bone xenograft, a bone allograft, and mixtures thereof, and
the outer layer comprises a carbonate salt.

2. The porous bone graft material according to claim 1, wherein the pores of the core have a surface which is at least partially coated with the carbonate salt.

3. The porous bone graft material according to claim 1, wherein the particles have a weight ratio of carbonate salt to core material from 15:100 to 1:100.

4. The porous bone graft material according to claim 1, wherein the alloplast is selected from the group consisting of hydroxy apatite, α-tricalcium phosphate, β-tricalcium phosphate, tetracalcium phosphate, dicalcium phosphate anhydrous, dicalcium phosphate dihydrate, amorphous calcium phosphate, calcium deficient hydroxyapatite, calcium sulphate and bioactive glass.

5. The porous bone graft material according to claim 1, wherein the core has a porosity of at least 50%.

6. The porous bone graft material according to claim 1, wherein the carbonate is selected from the group consisting of sodium hydrogen carbonate, ammonium hydrogen carbonate, sodium carbonate, calcium carbonate, strontium carbonate and magnesium carbonate.

7. The porous bone graft material according to claim 1, wherein the bone graft particles have an average particle size of 0.1 to 3.0 mm.

8. The porous bone graft material according to claim 1, wherein the bone graft particles have a mean pore size of 200-1000 μm.

9. A method for preparing a porous bone graft material by mixing an acid containing self-setting adhesive composition with bone graft particles at the onset of curing, wherein:
the bone graft particles comprise a porous core with an outer surface that is at least partially covered by an outer layer,
the core comprises a core material selected from the group consisting of an alloplast, a bone xenograft, a bone allograft, and mixtures thereof, and
the outer layer comprises a carbonate salt.

10. The method according to claim 9, wherein the acid containing self-setting adhesive composition comprises an aqueous solution and an acid containing self-setting adhesive powder, wherein the acid containing self-setting adhesive powder comprises at least a multivalent metal salt and phosphoserine.

11. A kit for preparing a porous bone graft material comprising
a) a first component A comprising an aqueous solution,
b) a second component B comprising bone graft particles, and
an acid containing self-setting adhesive powder that is present in the second component B or forms a third component C, wherein:
said acid containing self-setting adhesive powder comprises at least a multivalent metal salt and phosphoserine,
the bone graft particles comprise a porous core with an outer surface that is at least partially covered by an outer layer,
the core comprises a core material selected from the group consisting of an alloplast, a bone xenograft, a bone allograft, and mixtures thereof, and
the outer layer comprises a carbonate salt.

12. The kit according to claim 11, wherein the acid containing self-setting adhesive powder is present in the second component B.

13. The kit according to claim 11, wherein component A additionally comprises an acidifying agent selected from the group consisting of hydrochloric acid, ascorbic acid, citric acid, malic acid, tartaric acid, maleic acid, succinic acid, fumaric acid, acetic acid, formic acid and propionic acid or mixtures thereof.

14. The kit according to claim 11, wherein the acid containing self-setting adhesive powder additionally comprises calcium silicate.

15. A method for treating a bone defect comprising:
preparing a porous bone graft material by performing the method according to claim 9, and
applying the porous bone graft material to the site of the bone defect directly after mixing the bone graft particles with the acid containing self-setting adhesive composition to treat the bone defect.

16. The kit according to claim 11, wherein the acid containing self-setting adhesive powder forms the third component C.
